(19) United States
(12) Patent Application Publication
(10) Pub. No.: US 2009/0074860 A1
(43) Pub. Date: Mar. 19, 2009

(54) NUTRITIONAL BEVERAGE WITH SKIN IMPROVEMENT AIDS

(76) Inventor: Scott Vincent Borba, Oxnard, CA (US)

Correspondence Address: LACKENBACH SIEGEL, LLP, LACKENBACH SIEGEL BUILDING, 1 CHASE ROAD, SCARSDALE, NY, 10583, US

(21) Appl. No.: 11/719,183

(22) PCT Filed: Nov. 15, 2005
(86) PCT No.: PCT/US05/41342

§ 371 Date: May 11, 2007

Related U.S. Application Data
(60) Provisional application No. 60/628,057, filed on Nov. 15, 2004.
(60) Provisional application No. 60/628,068, filed on Nov. 15, 2004.
(60) Provisional application No. 60/628,059, filed on Nov. 15, 2004.

(51) Int. Cl. A23L 1/302; A23L 1/303; A61K 9/20

(52) U.S. Cl. 424/464; 426/72; 426/73

(57) ABSTRACT

This invention provides a nutritional beverage solution assistive to the visual appearance of a consumer's skin. In optional preferred embodiments, the solution includes primary antioxidant Vitamins C & E, secondary antioxidant of at least one of a Grape Seed Extract, a Papaya Extract, a Guava Extract, an Acai Extract, a Green Tea Extract, a Chamomile Extract, a Cherimoya Extract, a Longan Extract, a Lychee Extract, a Pomegranate Extract, a Walnut Extract or a Mangosteen Extract, along with a unique combination of other select ingredients to improve the nutritional content, and consumer acceptance of the beverage including taste and color factors. Alternative embodiments may vary the ingredients and may be placed be in a variety of consumer desired forms including paste, powder, gel, or encapsulated in a pill.

### RELATED APPLICATIONS

This application claims priority from U.S. Prov. App. No. 60/628,058 filed Nov. 15, 2004, U.S. Prov. App. No. 60/628,057 filed Nov. 15, 2004, and U.S. Prov. App. No. 60/628,059 filed Nov. 15, 2004, the entire contents of each of which are fully incorporated herein by reference.

### BACKGROUND OF THE INVENTION

1. Field of the Invention

The present invention relates to a nutritional beverage in ready-to-drink, powder, and other convenient forms. More specifically, the present invention relates to a beverage formulation to improve skin moisture, elasticity, fine lines, dryness, roughness, firmness, complexion (breakouts), hydration, texture and redness.

2. Description of the Related Art

It is well known that certain vitamins, minerals and botanical ingredients contain antioxidants. Antioxidants are chemicals that protect cells by neutralizing external forces or the effects of such external forces (such as damage from the sun, pollution, wind, and temperature) and internal factors (for example, emotions, metabolism, and the presence of excess oxygen).

It is also known in the medical field that antioxidants assist in skin repair and the strengthening of blood vessels because they combat free radicals. Free radicals are byproducts that are formed when oxygen is used by the body.

Vitamin E is the most abundant fat-soluble antioxidant in the body. It is one of the most efficient chain-breaking antioxidants available, and serves as a primary defender against detrimental oxidation. Vitamin E is also a primary defender against lipid peroxidation (creation of unstable molecules containing more oxygen than is usual).

Vitamin C is the most abundant water-soluble antioxidant in the body, and acts primarily in cellular fluid.

A wide variety of other beverage ingredients are known to those skilled in the art of formulating sports drinks, beverages, and other consumer products. Unfortunately, the problem with other types of nutritional beverages is that none is formulated, nor clinically tested, to specifically address skin conditions employing the formulation of the present invention and as a consequence, the present invention achieves an important consumer benefit and advantage.

It is also known to use fruit from the lychee tree to create a variety of foods and beverages. The fruit is believed to have various health benefits not fully understood at this time. Common lychee-products include lychee fruit, jam, juice, tee, sauce, and alcohol mixtures (martinis etc.).

It is further known to select nutritional and medical aids to avoid adult acne or skin blemish concerns. Common skin aids for harnessing antioxidants include pomegranate extracts and other common fruit extracts. Walnut extract has also been suggested as providing a skin assistive benefit.

A wide variety of other beverage ingredients are known to those skilled in the art of formulating sports drinks, age-defying beverages, and other consumer products.

Finally, It is well known to select nutritional and diet aids in an attempt to resist the effects of skin aging, namely wrinkles, and sagging. Grape seed and Acai berry extract has been suggested as providing a skin assistive benefit when mixed with other antioxidants in juices.

Unfortunately, the problem with other types of nutritional beverages is that none is formulated to specifically address skin balancing, skin clarifying, or skin renewal or other skin benefits; employing the respective formulations of the present invention and as a consequence, the present invention achieves an important consumer benefit and advantage.

### OBJECTS AND SUMMARY OF THE INVENTION

An object of the present invention is to provide a nutritional beverage in ready to drink powder forms to consumers to improve at least one of a group of skin concerns including: skin moisture, elasticity, fine lines, dryness, roughness, firmness, complexion (breakouts), hydration, texture and redness.

Another object of the present invention is to provide the product in a form that is visually attractive and has a pleasing taste to the consumer.

The present invention broadly relates to a nutritional beverage solution assistive to the visual appearance of a consumer's skin. In optional preferred embodiments, the solution includes primary antioxidant Vitamins C & E, secondary antioxidant of at least one of a Grape Seed Extract, a Papaya Extract, a Guava Extract, an Acai Extract, a Green Tea Extract, a Chamomile Extract, a Cherimoya Extract, a Longan Extract, a Lychee Extract, a Pomegranate Extract, a Walnut Extract or a Mangosteen Extract, along with a unique combination of other select ingredients to improve the nutritional content, and consumer acceptance of the beverage including taste and color factors. Alternative embodiments may vary the ingredients and may be placed be in a variety of consumer desired forms including paste, powder, gel, or encapsulated in a pill.

According to one alternative embodiment of the present invention, there is provided a nutritional beverage solution comprising primary antioxidants Vitamin C and Vitamin E, as well as a secondary antioxidant of at least one of a Grape Seed Extract, a Papaya Extract, a Guava Extract, an Acai Extract, a Green Tea Extract, a Chamomile Extract, a Cherimoya Extract, a Longan Extract, a Lychee Extract, a Pomegranate Extract, a Walnut Extract or a Mangosteen Extract. Natural or artificial sweeteners, colors and flavors are also included for good taste and appearance.

According to another alternative embodiment of the present invention there is provided a nutritional beverage solution comprising water, tocopheryl acetate, magnesium gluconate, pantothenic acid, potassium, ascorbic acid, niacinamide, zinc, retinyl palmitate, pyridoxine HCL, calcium sulfate dehydrate, phytonadione, cyanocobalamin, selenium amino acid, and at least one of a grape seed extract, a sage leaf extract, a papaya extract, a guava extract, a acai extract, a green tea extract, a chamomile extract, and a lychee extract.

According to another alternative embodiment of the present invention there is provided a nutritional beverage solution comprising, purified water, tocopheryl acetate, magnesium gluconate, pantothenic acid, potassium, ascorbic acid, niacinamide, zinc, retinyl palmitate, pyridoxine HCL, calcium sulfate dehydrate, phytonadione, walnut extract, selenium amino acid, gelatin, pomegranate extract, and optionally cyanocobalamin.

According to another alternative embodiment of the present invention there is provided a nutritional beverage solution comprising, purified water, tocopheryl acetate, magnesium gluconate, pantothenic acid, grape seed extract, potassium, ascorbic acid, niacinamide, zinc, retinyl palmitate, pyridoxine HCL, calcium sulfate dehydrate, phytonadione, green tea extract, chamomile extract, cyanocobalamin, selenium amino acid, and acai extract.

According to another optional and one alternative embodiment of the present invention there is provided a powder comprising: DL-alpha Tocopheryl Acetate, Magnesium Gluconate USP, D-Calcium Pantothenate USP, Potassium Phosphate Dibasic USP, Ascorbic Acid USP, Niacinamide USP, Zinc Picolinate, Vitamin A, Pyrodoxine HCL USP, Calcium Sulfate Dihydrate, Vitamin K-1, Vitamin B-12, and Selenium AA Complex.

According to another embodiment of the present invention, there is provided a powdered mixture comprising the ingredients alternatively noted as above, in addition to binding agents such as Dextrose, Trical Phosphate or Silicon Dioxide.

According to another embodiment of the present invention, the alternatively noted compositions may additionally include artificial and natural colors, sweeteners, and flavors in combinations suitable to enhance consuming enjoyment.

The above, and other objects, features and advantages of the present invention will become apparent from the following description read in conjunction with the accompanying drawings, in which like reference numerals designate the same elements.

### BRIEF DESCRIPTION OF THE DRAWINGS

None

### DETAILED DESCRIPTION OF THE PREFERRED EMBODIMENTS

In coping with the problems noted above, the present invention provides a nutritional beverage solution containing Vitamins E & C as primary antioxidants.

Beverage embodiments may additionally includes a secondary antioxidant of at least one of a Grape Seed Extract, a Papaya Extract, a Guava Extract, an Acai Extract, a Green Tea Extract, a Chamomile Extract, a Cherimoya Extract, a Longan Extract, a Lychee Extract, a Pomegranate Extract, a Walnut Extract or a Mangosteen Extract, in addition to other specially selected ingredients to ensure pleasant consumer taste, color, and fluid texture or viscosity.

Select embodiments of the present invention may also provide a nutritional beverage solution containing secondary antioxidants as extracts solely from the Lychee tree fruit (Lychee tree fruit has a high vitamin C per gram concentration), extracts from the pomegranate fruit and walnut fruit, or extracts from the acai fruit or berry and grape seeds.

In sum, the present beverage includes a carefully balanced combination of natural skin assisting agents, other vitamins, minerals, skin hydration components, and other factors to ensure pleasant consumer taste, color, and fluid texture or viscosity while ensuring delivery of the positive effects noted herein.

In another alternative embodiment of the present invention, the nutritional beverage solution further contains sucralose, natural flavors, calcium sulfate, sodium benzoate, potassium sorbate FCC, and at least one of FD&C Blue No. 1, FD&C Yellow No. 5, for consumer desired beverage color, taste, texture, and preservation, and optionally malic acid.

In another alternative embodiment of the present invention, the nutritional beverage solution further contains sucralose, natural flavors, calcium sulfate, sodium benzoate, and at least one of FD&C Red. No. 40, FD&C Yellow No. 5, and malic acid for consumer desired beverage color, taste, and texture.

In another alternative embodiment of the present invention, the nutritional beverage solution further contains sucralose, natural flavors, calcium sulfate, sodium benzoate, and at least one of FD&C Red. No. 40, FD&C Blue No. 1, for consumer desired beverage color, taste, and texture, and in further optional (not required) embodiments malic acid.

In another alternative embodiment, the above general nutritional beverage solution mixture may be alternatively provided in a dry powdered form suitable for mixing with a selected beverage and may additionally include flavors, colors, and other binding ingredients.

Referring now to a variety of ingredient solutions described hereinafter, those skilled in the art will understand that for the present invention, a powdered solution may provided suitable for mixing with 15.2-20 FL OZ of a preferred liquid (water, fruit juice, etc.) and as shown includes selected additional extracts, flavors, sweeteners, and preservatives to provide additional benefits. While the tables below provide an approximate amount for mixing into a common consumer container, the serving size is obviously regulated to 8.00 FL OZ (240 ml) according to government requirement. It will be obvious to those skilled in the art, that as disclosed herein, the present invention may be scaled-up or downwardly dependent upon volume and a desired manufacturers convenience as long as the general ratios (powder/fluid) suggested below are reasonably maintained. It should also be understood, that due to the substantial dilution during mixing, any one or more of the secondary antioxidant ingredients listed in the tables may be dropped altogether or an alternative ingredient may be substituted.

While the mixture ratios provided in FIG. 1 (ingredient weight/total weight in powdered form) are preferred, the present invention also envisions a range of ingredient weights for alternative embodiments depending upon a desired vitamin, mineral, flavor count, or for other reasons including advertising advantage. For example, the following Table I includes proposed ranges suitable for mixing as noted above into 15.2 to 20 FL OZ solutions presently believed to aid in skin health and replenish consumed skin nutrients providing an improved healthy look to the skin.

TABLE I

| Ingredient | Per Mixture |
|---|---|
| Primary Antioxidant Vitamin E | 50-360 mg |
| Primary Antioxidant Vitamin C | 25-100 mg |
| Secondary Antioxidant Grape Seed Extract, | 25-100 mg |
| Papaya Extract, Guava Extract, Acai Extract, | |
| Green Tea Extract, Chamomile Extract, Cherimoya | |
| Extract, Longan Extract, Lychee Extract, a Pomegranate | |
| Extract, a Walnut Extract, or a Mangosteen | |
| Extract, and other Secondary Antioxidants | |

As a consequence of the above-proposed alternative solutions, the present invention makes available a consumable product that may be easily adapted to a gel or a paste delivery system by removal of water and/or the addition of food-based structural aids (allowing the easy formation of a gel or paste).

In another alternative embodiment of the present invention, selective combinations may optionally or additionally include any one of Sage Leaf Extract, Yerba Mate Extract or Soy.

In another alternative embodiment of the present invention Vitamin A Palmitate (Vitamin A) is optionally and alternatively provided above in Table 1 in a weight of approximately 2500 IU +/−0.05, a substantial increase for selective embodiments.

In another alternative embodiment of the present invention Nicacinamide (Niacin) is optionally and alternatively provided above in Table 1 in a weight of approximately 24 mg +/−0.05, a substantial increase for selective embodiments.

In another alternative embodiment of the present invention Pridoxine HCI (Vitamin B6) is optionally and alternatively provided above in Table 1 in a weight of approximately 7.3 mg +/−0.05, a substantial increase for selective embodiments.

In another alternative embodiment of the present invention Vitamin B12 is optionally and alternatively provided above in Table 1 in a weight of approximately 0.05 mg +/−0.05, a substantial increase for selective embodiments.

In another alternative embodiment of the present invention D-Calcium Pantothenate (Panothenic Acid) is optionally and alternatively provided above in Table 1 in a weight of approximately 20.6 mg +/−0.05, a substantial increase for selective embodiments.

In another alternative embodiment of the present invention Calcium Sulfate Dihydrate (Calcium) is optionally and alternatively provided above in Table 1 in a weight of approximately 2 mg +/−0.05, a substantial increase for selective embodiments.

In another alternative embodiment of the present invention Magnesium Gloconate (Magnesium) is optionally and alternatively provided above in Table 1 in a weight of approximately 240 mg +/−0.05, a substantial increase for selective embodiments.

In another alternative embodiment of the present invention Zinc Picolinate (Zinc) is optionally and alternatively provided above in Table 1 in a weight of approximately 10 mg +/−0.05, a substantial increase for selective embodiments.

In another alternative embodiment of the present invention Selenium AA

Complex (Selenium) is optionally and alternatively provided above in Table 1 in a weight of approximately 0.04 mg +/−0.05, a substantial increase for selective embodiments.

In another alternative embodiment of the present invention Potassium Phosphate Dibasic (Potassium) is optionally and alternatively provided above in Table 1 in a weight of approximately 0.10 mg +/−0.05, a substantial increase for selective embodiments.

In another alternative embodiment of the present invention Vitamin K is optionally and alternatively provided above in Table 1 in a weight of approximately 50 mg +/−0.05, a substantial increase for selective embodiments.

Referring now to Tables II, through VII for alternative formulations of the present invention.

TABLE II
BORBA REPLENISHING INGREDIENTS
Serving Size 1 Bottle

| | Amount per Bottle | |
|---|---|---|
| Vitamin A | 2500 | IU |
| Vitamin C (as ascorbic acid) | 50 | mg |
| Vitamin E (as d-alpha tocopheryl acetate) | 300 | IU |
| Niacin (as niacinamide) | 24 | mg |
| Vitamin B6 (as pyroxidine HCl) | 7.3 | mg |
| Vitamin B12 | .05 | mg |
| Pantothenic Acid (as calcium | 20.6 | mg |
| pantothenate) | | |
| Calcium (as calcium sulfate) | 2 | mg |
| Magnesium (as magnesium gluconate) | 240 | mg |
| Zinc (as zinc piccolinate) | 10 | mg |
| Selenium (as Selenium AA Complex 1%) | .04 | mg |
| Potassium (as potassium phosphate) | 50 | mg |
| Vitamin K | .01 | mg |
| Extract Blend | | |

Grape Seed Extract (50 mg), Green Tea Extract (100 mg), Chamomille Extract (25 mg), Acai Extract (25 mg), Sage Leaf Extract (25 mg), Payapa Extract (25 mg), Guava Extract (25 mg), Lychee Extract (25 mg)

TABLE III
BORBA CLARIFYING INGREDIENTS
Serving Size 1 Bottle

| | Amount per Bottle | |
|---|---|---|
| Vitamin A | 2500 | IU |
| Vitamin C (as ascorbic acid) | 55 | mg |
| Vitamin E (as d-alpha tocopheryl acetate) | 330 | IU |
| Niacin (as niacinamide) | 26.4 | mg |
| Vitamin B6 (as pyroxidine HCl) | 8 | mg |
| Vitamin B12 | .055 | mg |
| Pantothenic Acid (as calcium | 22.7 | mg |
| pantothenate) | | |
| Calcium (as calcium sulfate) | 2 | mg |
| Magnesium (as magnesium gluconate) | 264 | mg |
| Zinc (as zinc piccolinate) | 11 | mg |
| Selenium (as Selenium AA Complex 1%) | .044 | mg |
| Potassium (as potassium phosphate) | 55 | mg |
| Vitamin K | .011 | mg |

| | |
|---|---|
| Extract Blend | * |

Walnut Powder (25 mg), Pomegranate Extract (25 mg)

TABLE IV
BORBA AGE DEFYING INGREDIENTS
Serving Size 1 Bottle

| | Amount per Bottle | |
|---|---|---|
| Vitamin A | 2500 | IU |
| Vitamin C (as ascorbic acid) | 60 | mg |
| Vitamin E (as d-alpha tocopheryl acetate) | 360 | IU |
| Niacin (as niacinamide) | 28.8 | mg |
| Vitamin B6 (as pyroxidine HCl) | 8.760 | mg |
| Vitamin B12 | .06 | mg |
| Pantothenic Acid (as calcium | 24.7 | mg |
| pantothenate) | | |
| Calcium (as calcium sulfate) | 2 | mg |
| Magnesium (as magnesium gluconate) | 288 | mg |
| Zinc (as zinc piccolinate) | 12 | mg |
| Selenium (as Selenium AA Complex 1%) | .0480 | mg |
| Potassium (as potassium phosphate) | 60 | mg |
| Vitamin K | .012 | mg |
| Extract Blend | | |

Grape Seed Extract (50 mg), Green Tea Extract (100 mg), Chamomille Extract (25 mg), Acai Extract (25 mg)

TABLE V
BORBA CALMING INGREDIENTS
Serving Size 1 Bottle

| | Amount per Bottle | |
|---|---|---|
| Vitamin A | 2525 | IU |
| Vitamin C (as ascorbic acid) | 60.6 | mg |
| Vitamin E (as d-alpha tocopheryl acetate) | 363.6 | IU |
| Niacin (as niacinamide) | 29.09 | mg |
| Vitamin B6 (as pyroxidine HCl) | 8.85 | mg |
| Vitamin B12 | .061 | mg |
| Pantothenic Acid (as calcium | 24.95 | mg |
| pantothenate) | | |
| Calcium (as calcium sulfate) | 2.02 | mg |
| Magnesium (as magnesium gluconate) | 290.88 | mg |
| Zinc (as zinc piccolinate) | 12.12 | mg |
| Selenium (as Selenium AA Complex 1%) | 0.0485 | mg |
| Potassium (as potassium phosphate) | 60.6 | mg |
| Vitamin K | .01212 | mg |
| Extract Blend | | |

Grape seed extract (26 mg), Green tea extract (104 mg), Chamomille extract (26 mg), Acai extract (26 mg) Longan extract (50 mg).

TABLE VI
BORBA FIRMING INGREDIENTS
Serving Size 1 Bottle

| | Amount per Bottle | |
|---|---|---|
| Vitamin A | 2550 | IU |
| Vitamin C (as ascorbic acid) | 61.2 | mg |
| Vitamin E (as d-alpha tocopheryl acetate) | 429.624 | IU |
| Niacin (as niacinamide) | 29.376 | mg |
| Vitamin B6 (as pyroxidine HCl) | 8.935 | mg |
| Vitamin B12 | .0612 | mg |
| Pantothenic Acid (as calcium | 25.194 | mg |
| pantothenate) | | |
| Calcium (as calcium sulfate) | 2.04 | mg |
| Magnesium (as magnesium gluconate) | 293.76 | mg |
| Zinc (as zinc piccolinate) | 12.24 | mg |
| Selenium (as Selenium AA Complex 1%) | 0.04896 | mg |
| Potassium (as potassium phosphate) | 61.2 | mg |
| Vitamin K | .01224 | mg |
| Extract Blend | | |

Grape seed extract (51 mg), Green tea extract (102 mg), Chamomille extract (25.5 mg), Acai extract (25.5 mg), Cherrimoya extract(25 mg), Yerba mate extract (4 mg), soy bean (25 mg)

TABLE VII
BORBA ANTI-AGE/CLARIFYING INGREDIENTS
Serving Size 1 Bottle

| | Amount per Bottle | |
|---|---|---|
| Vitamin A | 2575 | IU |
| Vitamin C (as ascorbic acid) | 61.8 | mg |
| Vitamin E (as d-alpha tocopheryl acetate) | 370.8 | IU |
| Niacin (as niacinamide) | 29.66 | mg |
| Vitamin B6 (as pyroxidine HCl) | 9.023 | mg |
| Vitamin B12 | .0618 | mg |
| Pantothenic Acid (as calcium | 25.44 | mg |
| pantothenate) | | |
| Calcium (as calcium sulfate) | 2.06 | mg |
| Magnesium (as magnesium gluconate) | 298.76 | mg |
| Zinc (as zinc piccolinate) | 12.36 | mg |
| Selenium (as Selenium AA Complex 1%) | 0.0494 | mg |
| Potassium (as potassium phosphate) | 61.8 | mg |
| Vitamin K | .01236 | mg |
| Extract Blend | | |

Grape seed extract (53.3 mg), Gree tea extract (106.6 mg) Chamomille extract (26.65 mg), Acai extract (26.65 mg), Mangosteen extract (1.25 mg)

A number of tests were run on each of the compositions noted above in tables II through VII, with the following results as summarized below. In each case test subjects were provided with the combination as presented above in contrast with a control group. Analysis was conducted visually and employing survey techniques common in the skin care and cosmetics trade, allowing support for the generation of the claims noted below.

(A) Age Defying—Scientifically proven to slow the visible signs of aging by an average of 30%. Fights signs of premature aging, aids rejuvenating of aging skin, and helps slow the visible signs of aging in just 7 days.
(B) Clarifying—Independent respondents reported a reduction in breakouts by an average of 52% of respondents reporting a reduction in breakouts. Helps prevent and reduce blemishes, promotes healthy skin, and generates a visual skin clearing benefits in just 28 days.
(C) Replenishing—Scientifically proven to improve moisture levels by an average of 65%. Keeps skin hydrated, improves skin texture, and improves skin's natural moisture levels in just 7 days.
(D) Firming—Scientifically proven to improve elasticity by an average of 24%. Benefits inclusion of a fat converter, fat burner and antioxidant, hormonal balancing, and visible skin firming benefits in just 7 days.
(E) Calming-Clinical research indicates a reduction of redness up to 61%. Benefits are helps reduce irritation and redness, improves texture and tone, and helps promote more tranquil skin in just 28 days.
(F) Anti-Age/Blemish-Scientifically proven to improve skin clarity and slow signs of aging by an average of 16%. Benefits include improve clarity, minimizes signs of aging, and improves skin's clarity and signs of aging in just 7 days.

Clinical Results for Age-Defying/Replenishing/Firming/Calming/Anti-Age & Clarifying Formulations:

The objective of each clinical study was to assess the efficacy of fluid combinations to improve the appearance of the skin with respect to dryness, roughness, fine lines, elasticity/firmness/resiliency, skin clarity and subject perception of skin condition based against a starting base line and a defined index of the parameter in question.

Visual assessments were made by an experienced technician at the baseline interval and repeated at the conclusion of the study relative to a predetermined index of measurement. A dermatologist evaluated irritation, as erythema, at the baseline visit and at the conclusion.

### Skin Dryness

Treatment of skin with Skin Balance Water significantly reduced the level of dryness by Week 1 and persisted through Week 4. In contrast, no significant improvement was detected in the untreated control group as a function of time. Comparatively, the test group out-performed the control group at the Week 4 evaluation interval.

### Superficial Fine Lines/Wrinkles

Treatment of skin with the test product numerically improved the appearance of fine lines/wrinkles at the Week 1 evaluation interval and significantly improved the lines/wrinkles at the Week 4 assessment interval. In contrast, no significant improvement was detected in the untreated control group as a function of time. Comparatively, no significant performance differences between groups were detected. However, a greater level of improvement was noted in the test group as compared to the control group at both Week 1 and Week 4, suggesting a product-induced effect.

### Skin Roughness

Treatment of skin with Skin Balance Water numerically improved skin roughness by the Week 1 interval and significantly improved the roughness at the Week 4 assessment interval. In contrast, no significant improvement was detected in the untreated control group as a function of time. Comparatively, the test group out-performed the control group at the Week 4 evaluation interval.

### Skin Elasticity

Treatment of skin with the test product significantly improved skin elasticity by the Week 1 interval and persisted through to Week 4. Similarly, improvement was also noted at both the Week 1 and 4 assessment intervals. Numerically, a greater level of improvement was observed in the test group as compared to the control group and the test group significantly out-performed the control group at Week 4.

### Skin Clarity

Significant improvement in skin clarity was observed at the Week 1 and 4 intervals as a function of treatment with Skin Balance Water. Significant improvement was also detected in the control group at Week 4 only. Comparatively, Skin Balance Water significantly out-performed the control group at both the Week 1 and 4 assessment intervals.

### Skin Irritation

Decreased signs of skin irritation were detected in the treatment group, as compared to the control group, over the course of the study.

Under the conditions employed in the study, treatment of skin with Skin Balance compositions significantly improved skin condition relative to baseline values relative to the visual index, and the untreated control group. Significant improvement was detected in the following parameters: skin dryness, roughness, elasticity and clarity.

Numerically, the test product out-performed the control group with respect to fine lines/wrinkles suggesting a product-induced effect. With respect to subjective perception of product performance, at least 50% of the panelists indicated improvement in skin condition in five (5) of the seven (7) parameters evaluated, with a range of improvement from 38.67% to 50.63%. Subjective results were as follows:

TABLE VIII

| | | % improvement |
|---|---|---|
| | % of panel | indicated by panel |
| Question* | answering “Yes” | that responded “Yes” |
| Reduced Skin Dryness ? | 59.3% | 46.9% |
| Reduced Skin Roughness ? | 59.3% | 47.5% |
| Improved Fine Lines ? | 40.7% | 41.81% |
| Improved Elasticity ? | 51.9% | 41.43% |
| Improved Firmness ? | 55.6% | 38.67% |
| Improved Clarity ? | 37.0% | 50.0% |
| Improved Complexion ? | 59.3% | 50.63% |

Below are the percent improvement figures from evaluation performed by the clinical technician. Instances where the Baseline vs. Week 1 or Week 4 mean results are significant are noted with a “*”. Instances where the Skin Balance Water differences from Baseline were significantly different than control are noted with a “p”.

TABLE VIII

| | Week 1 | Week 1 | Week 4 | Week 4 |
|---|---|---|---|---|
| | Treatment | Control | Treatment | Control |
| Dryness | 52.9%* | 26.9% | 33.8%* p | 19.2% |
| Fine Lines | 3.79% | 1.66% | 11.39%* | 4.07% |
| Roughness | 8.31% | 5.30% | 29.98%* p | 11/09% |
| Elasticity | 14.98%* | 10.87%* | 23.83%* p | 9.46%* |
| Clarity | 18.87%* p | 5.93% | 22.47%* p | 7.38% |
| (worse) | | | | |
| Erythema | Not Evaluated | Not Evaluated | 60.98% | 0.00% |

### Clinical Results

### Clarifying

The objective of this study was to evaluate the acnegenic/comedogenic potential of Skin Balance Water Composition, clarifying by quantifying a decrease or an increase in signs of acne.

A dermatologist conducted a global evaluation of acne at the Baseline (Day 0) and Week 4 visits by counting all non-inflammatory (open and closed comedones) and inflammatory lesions (papules and pustules) and cysts. Each lesion type was counted and recorded separately.

After four (4) weeks of use of Skin Balance Water—Clarifying, a trendwise reduction in open comedones was observed relative to baseline values. In contrast, no significant changes in non-inflammatory lesions were noted as a function of time. Comparatively, a trendwise and significantly greater reduction in open comedones was detected in the test group relative to the control group.

Under the conditions employed in this study, a significant decrease in open comedones was detected in the Skin Balance Water Composition—Clarifying group after four (4) weeks of use relative to baseline values and the untreated control group. No other significant changes were observed in either non-inflammatory or inflammatory lesions as a function of treatment or time. Use of the test product was well tolerated as no significant changes in irritation were detected in the test group as a function of treatment or time. With regard to the subjective perception of the performance of Skin Balance Water Composition—Clarifying, more subjects indicated positive responses to questions regarding performance than negative responses.

The subjective results for Clarifying are as follows:

TABLE IX

| | % of panel | % improvement |
|---|---|---|
| | answering | indicated by panel |
| Question* | “Yes” | that responded “Yes” |
| Did use of the test product either | 31.0% | 41.1% |
| actual or perceived, help your | | |
| complexion? | | |
| Did the product help prevent | 37.9% | 52.7% |
| “breakouts”? | | |
| Did the product help to hydrate | 65.5% | 52.6% |
| your skin? | | |
| Did the product help keep facial | 44.8% | 50.8% |
| pores clear? | | |
| Did use of the product help skin | 58.6% | 48.8% |
| feel conditioned? | | |
| Did use of the product moisturize | 55.2% | 56.25% |
| your skin? | | |

In another alternative embodiment of the present invention the powder is lyophilized, meaning processing is a three-phase cycle to freeze, extract mobile water, then dry in controlled chambers. A carefully performed process forms lyophilized liquid activated powder.

It is further suggested that the present invention provides a ‘nutraceutical’ beverage, wherein the phrase ‘nutraceutical’ is a combination of ‘nutritional’ and ‘pharmaceutical’ and refers to foods that act as medicines or may have a suggested medical benefit or nutrition-focused benefit.

Having described at least one of the preferred embodiments of the present invention with reference to the accompanying drawings, it is to be understood that the invention is not limited to those precise embodiments, and that various changes, modifications, and adaptations may be effected therein by one skilled in the art without departing from the scope or spirit of the invention as defined in the appended claims.

What is claimed is:

1. A nutritional beverage combination for improving at least one of skin moisture, elasticity, fine lines, dryness, roughness, firmness, complexion (breakouts), hydration, texture and redness, the nutritional beverage comprising:
a primary antioxidant component;
said primary antioxidant component further comprising at least vitamin C and vitamin E in an amount sufficient to inhibit the activity of collagenase and elastase;
a secondary antioxidant component; and
said secondary antioxidant component further comprising at least one selected from the group consisting of a Grape Seed Extract, a Papaya Extract, a Guava Extract, an Acai Extract, a Green Tea Extract, a Chamomile Extract, a Cherimoya Extract, a Longan Extract, a Lychee Extract, a Pomegranate Extract, a Walnut Extract or a Mangosteen Extract, whereby said nutritional beverage aids slain improvement.

2. A nutritional beverage combination, according to claim 1, wherein:
said secondary antioxidant component further comprises at least one selected from the group consisting of Magnesium Gluconate USP, D-Calcium Pantothenate USP, Potassium Phosphate Dibasic USP, Niacinamide USP, Zinc Picolinate, Vitamin A, Pyrodoxine HCL USP, Calcium Sulfate Dihydrate, Vitamin K-1, Vitamin B-12, and Selenium AA Complex.

3. A nutritional beverage combination, according to claim 1, further comprising: at least one sweetener selected from a group consisting of an artificial sweetener, a natural sweetener, dextrose, maltitol, sucralose, and acesuflame potassium.

4. A nutritional beverage combination, according to claim 1, further comprising:
at least one of a natural and an artificial flavor.

5. A nutritional beverage combination, solution, according to claim 1, further comprising:
at least one of a natural and an artificial color.

6. A pill-based delivery system, comprising:
the combination according to claim 1 in the form of a reduced-water content gel.

7. A consumable paste delivery system, comprising:
the combination of claim 1, in the form of a reduced-water content paste.

8. A nutritional beverage combination, according to claim 1, further comprising:
a binding agent; and
said binding agent being at least one of dextrose, trical phosphate, and silicon dioxide

9. A nutritional beverage combination, according to claim 1, further comprising:
at least one of gelatin, sucralose, natural flavors, calcium sulfate, and sodium benzoate.

10. A nutritional beverage combination, according to claim 1, further comprising:
at least one of FD&C Blue No. 1, FD&C Yellow No. 5, and malic acid.

11. A nutritional beverage combination, comprising:
a primary antioxidant component;
said primary antioxidant component further comprising at least vitamin C and vitamin E in an amount sufficient to inhibit the activity of collagenase and elastase;
a secondary antioxidant component;
said secondary antioxidant component further comprising at least one selected from the group consisting of a Grape Seed Extract, a Papaya Extract, a Guava Extract, an Acai Extract, a Green Tea Extract, a Chamomile Extract, a Cherimoya Extract, a Longan Extract, a Lychee Extract, a Pomegranate Extract, a Walnut Extract or a Mangosteen Extract, whereby said nutritional beverage aids skin improvement;
a tertiary antioxidant component; and
said tertiary antioxidant component further comprising at least one selected from the group consisting of Magnesium Gluconate USP, D-Calcium Pantothenate USP, Potassium Phosphate Dibasic USP, Niacinamide USP, Zinc Picolinate, Vitamin A, Pyrodoxine HCL USP, Calcium Sulfate Dihydrate, Vitamin K-1, Vitamin B-12, and Selenium AA Complex.

12. A nutritional beverage, consisting essentially of:
a primary antioxidant component;
said primary antioxidant component further comprising at least vitamin C and vitamin E in an amount sufficient to inhibit the activity of collagenase and elastase; and
a secondary antioxidant component further comprising at least one selected from the group consisting of a Grape Seed Extract, a Papaya Extract, a Guava Extract, an Acai Extract, a Green Tea Extract, a Chamomile Extract, a Cherimoya Extract, a Longan Extract, a Lychee Extract, a Pomegranate Extract, a Walnut Extract or a Mangosteen Extract, whereby said nutritional beverage aids skin improvement.
